(19) United States
(12) Patent Application Publication
(10) Pub. No.: US 2007/0021758 A1
(43) Pub. Date: Jan. 25, 2007

(54) Anastomotic ring applier for use in colorectal applications

(75) Inventor: Mark S. Ortiz, Milford, OH (US)

Correspondence Address: FROST BROWN TODD, LLC, 2200 PNC CENTER, 201 E. FIFTH STREET, CINCINNATI, OH, 45202, US

(73) Assignee: Ethicon Endo-Surgery, Inc.

(21) Appl. No.: 11/187,658

(22) Filed: Jul. 22, 2005

(51) Int. Cl. A61B 17/08

(52) U.S. Cl. 606/153

(57) ABSTRACT

A surgical tool or applier operable to deploy an anastomotic ring comprises a handle connected to an elongate shaft. The elongate shaft comprises an anastomotic ring deployment mechanism on a distal portion of the shaft. The ring deployment mechanism is moveable from an unactuated position to an actuated position. The elongate shaft comprises a rigid, curved member, thereby rendering the tool applicable for colorectal applications. In one embodiment, the tool comprises an actuating mechanism that is operable to transfer an actuating force to the ring deployment mechanism via a flexible connecting member.

### FIELD OF THE INVENTION

The present invention relates, in general, to surgery and, more particularly, to a device for performing a surgical procedure on the digestive system.

### BACKGROUND OF THE INVENTION

The percentage of the world population suffering from morbid obesity is steadily increasing. Severely obese persons may be susceptible to increased risk of heart disease, stroke, diabetes, pulmonary disease, and accidents. Because of the effects of morbid obesity on the life of the patient, methods of treating morbid obesity have been the subject of intense research.

One known method for treating morbid obesity includes the use of anastomotic rings. Devices for applying anastomotic rings are known in the art. Devices of this nature are commonly adapted to insert a compressed anastomotic ring to an anastomotic opening formed between proximate gastrointestinal tissue walls. These applier devices may utilize a ring deployment mechanism comprising an expansion element that is actuated once the compressed ring is placed in the anastomotic opening, causing the anastomotic ring to expand from its compressed, cylindrically-shaped position to an actuated, hollow rivet-shaped position.

There may be circumstances in which it would be advantageous to have an anastomotic ring applier compatible for use in colorectal applications. However, the elongated shaft of a conventional anastomotic ring applier device may be unsuitable for colorectal applications. In particular, a conventional anastomotic ring applier device may lack a desirable curvature and/or may not provide ideal leverage for such applications. In addition, the actuating mechanism used by a conventional ring applier may be unsuitable for use in colorectal applications. A conventional ring applier may be unsuitable or otherwise less than ideal for colorectal applications for a variety of other reasons.

Consequently, it may be desirable to have an anastomotic ring applier device that is adapted for use with colorectal applications. Specifically, it may be desirable to have an anastomotic ring applier device that comprises an elongated shaft that is suited for colorectal applications. Further, it may be desirable to have an anastomotic ring applier device that includes an actuating mechanism suited for use in colorectal applications.

### BRIEF SUMMARY OF THE INVENTION

In one embodiment, an anastomotic ring applier device comprises a handle connected by an elongated shaft to a ring deployment mechanism configured to move an anastomotic ring from an unactuated, generally cylindrical position to an actuated, hollow rivet-forming position. The instrument further comprises an actuation mechanism for communicating an actuation force to the ring deployment mechanism. The elongate shaft comprises a rigid, curved element. The rigid, curved element of the elongate shaft may enable the instrument to be used in colorectal applications, or otherwise facilitate such use, to deploy an anastomotic ring.

In another embodiment, the instrument comprises a handle connected to a ring deployment mechanism by an elongate shaft comprising a proximal portion and a distal portion. The ring deployment mechanism comprises a plurality of fingers adapted to articulate outwardly from the elongate shaft to deploy an anastomotic ring. The elongate shaft comprises a rigid, curved member. This embodiment may also be suitable for use in colorectal applications.

In yet another embodiment, the instrument comprises a handle connected to a proximal portion of an elongate shaft, which further comprises a ring deployment mechanism located at a distal portion thereof. The ring deployment mechanism comprises a longitudinal end and a center portion, wherein the longitudinal end is adapted to move toward the center portion in order to deploy a portion of an anastomotic ring. The instrument further comprises an actuating mechanism that includes a flexible connecting member adapted to move the longitudinal end of the deployment mechanism toward the center portion. The elongate shaft comprises a rigid, curved member. This embodiment includes a rigid, curved component of the elongate shaft, as well as a flexible connecting member. This embodiment is thereby configured for use in deploying an anastomotic ring during colorectal applications.

### BRIEF DESCRIPTION OF THE FIGURES

The accompanying drawings, which are incorporated in and constitute a part of this specification, illustrate versions of the invention, and, together with the general description of the invention given above, and the detailed description of the versions given below, serve to explain the principles of the present invention.

FIG. 1 is a perspective view of an anastomotic ring applier device.

FIG. 2 is a partial perspective view of the distal portion of an anastomotic ring applier device holding an anastomotic ring in an unactuated position.

FIG. 3 is a partial perspective view of the distal portion of the device of FIG. 2 holding an anastomotic ring in the actuated position.

FIG. 4 is a frontal view of an actuated anastomotic ring.

FIG. 5 is a perspective view of the device of FIG. 1, shown with the ring deployment mechanism in the actuated position.

FIG. 6 is an exploded view of a distal portion of the device of FIG. 1, shown with the ring deployment mechanism fully actuated.

FIG. 7 is an exploded cross-sectional view of a proximal portion of the device of FIG. 1.

FIG. 8 is a cross-sectional view of a distal portion of the device of FIG. 1.

FIG. 9 is a cross-sectional view of a proximal portion of the device of FIG. 1.

FIG. 10 is a cross-sectional view of a distal portion of the device of FIG. 1, with the ring deployment mechanism fully actuated.

FIG. 11 is a cross-sectional view of a proximal portion of the device of FIG. 1, with the actuating members in the actuated position.

FIG. 12 is a cross-sectional view of a proximal portion of the device of FIG. 1, taken along plane 12 of FIG. 9.

### DETAILED DESCRIPTION OF EMBODIMENTS OF THE INVENTION

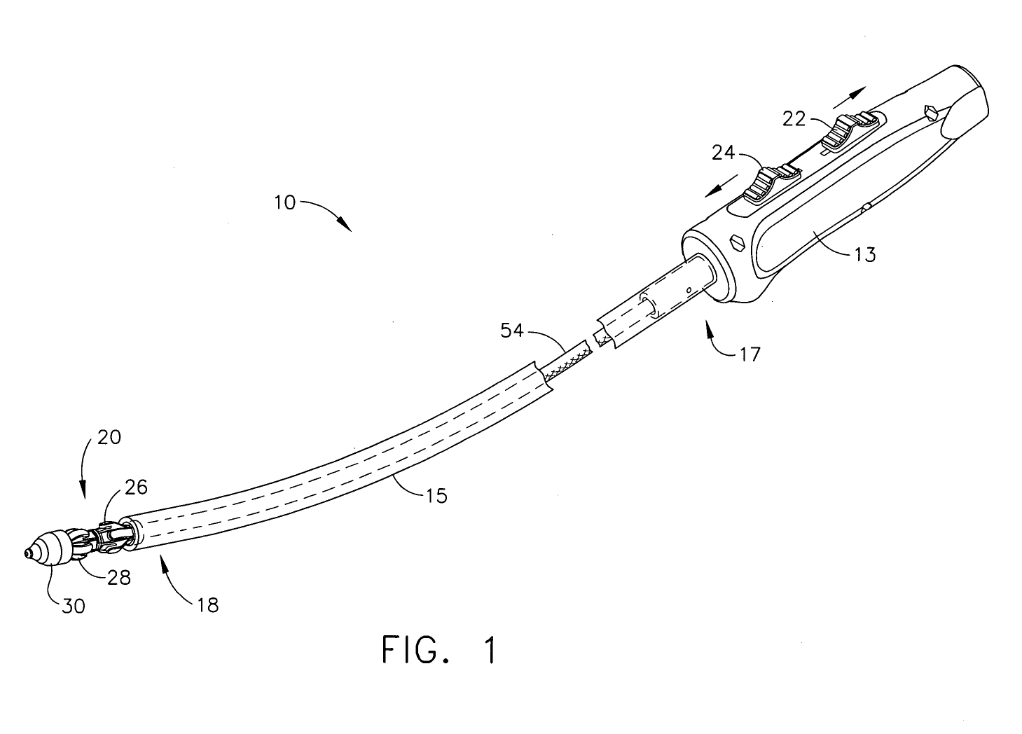

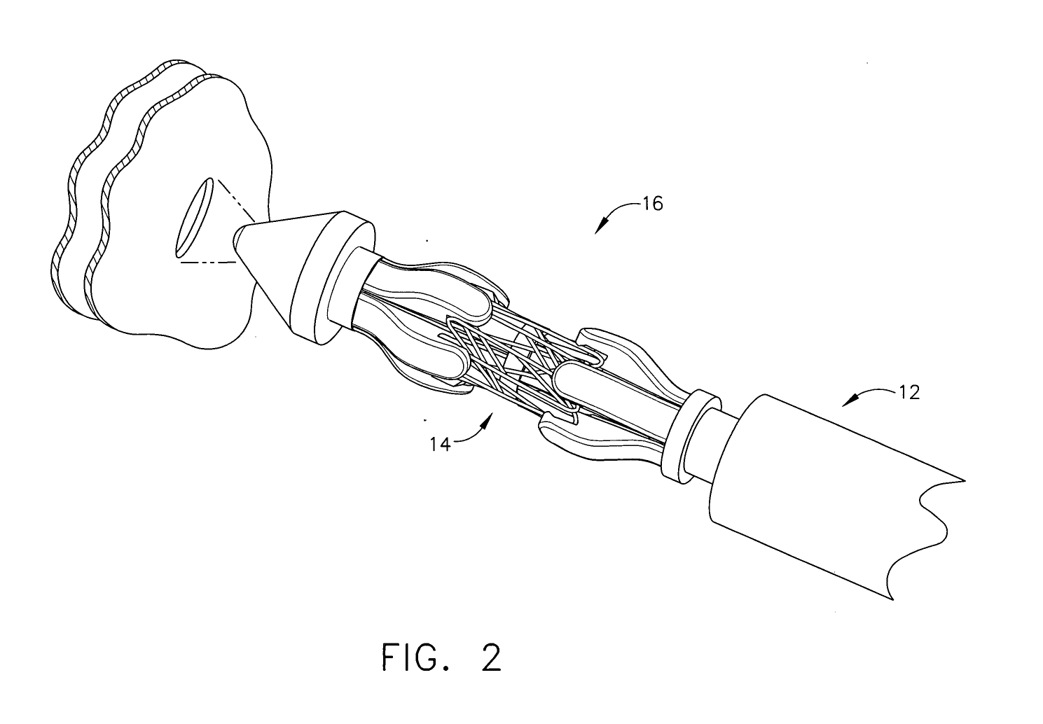

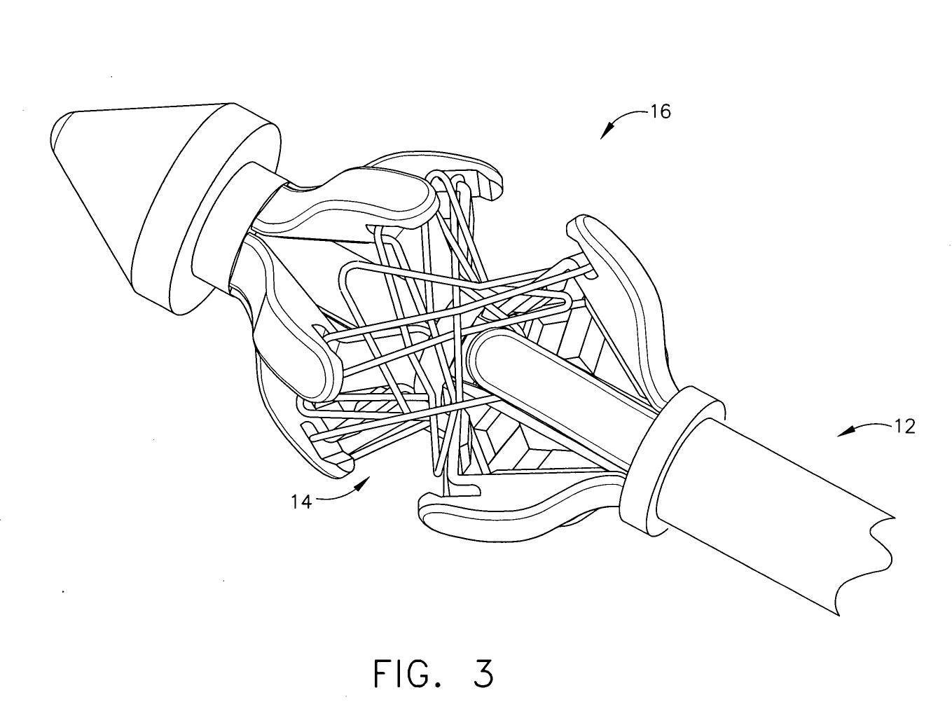

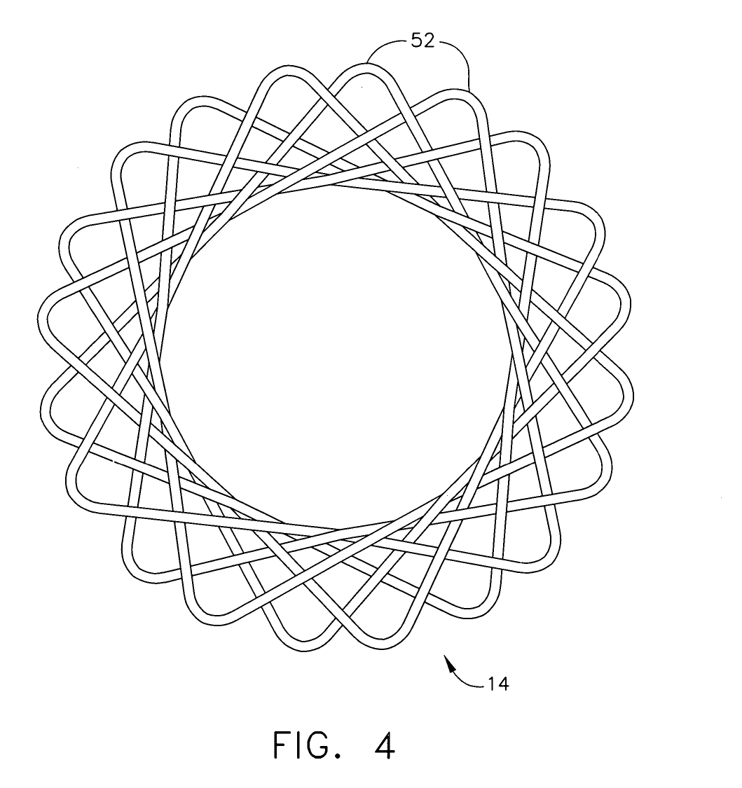

Turning to the Drawings, wherein like numerals denote like components throughout the several views, FIG. 1 depicts an applier 10 that is operable to deploy and actuate an anastomotic ring device (not pictured in FIG. 1) from a generally cylindrical shape to one having properties of a hollow rivet, or ring, capable of forming an anastomotic attachment at an anastomosis target site, such as in a bariatric gastric bypass of a morbidly obese patient. FIG. 2 depicts another applier 12. It will be appreciated that appliers 10, 12 may be used in a variety of ways, including but not limited to laparoscopically or endoscopically. Applier 12 is shown in FIG. 2 with an anastomotic ring 14 on a deployment mechanism 16. In FIG. 2, anastomotic ring 14 is shown in the compressed, cylindrically-shaped position. In FIG. 3, deployment mechanism 16 of applier 12 has moved anastomotic ring 14 to the actuated, hollow rivet-shaped position. FIG. 4 is a close-up view of anastomotic ring 14 in the actuated position. Anastomotic ring 14 may comprise a shape memory effect (SME) material, such as nitinol by way of example only, that further assists in actuation to an engaging hollow rivet shape. Other suitable anastomotic ring 14 materials will be apparent to those of ordinary skill in the art. An exemplary anastomotic ring 14 is described in detail in U.S. Patent Application Publ. No. US 2003/0032967 to Park et al.

It will be appreciated that the terms “proximal” and “distal” are used herein with reference to a clinician gripping a handle of applier 10. It will be further appreciated that for convenience and clarity, spatial terms such as “right”, “left”, “vertical” and “horizontal” are used herein with respect to the drawings. However, surgical instruments are used in many orientations and positions, and these terms are not intended to be limiting and absolute. In addition, aspects of the invention have application to surgical procedures performed endoscopically and laparoscopically, as well as an open procedure or other procedures. Use herein of one of these or similar terms should not be construed to limit the present invention for use in only one category of surgical procedure.

Referring to FIGS. 1, 5, and 6, applier 10 comprises a handle 13 and an elongated shaft 15 having a proximal end 17 and a distal end 18. Handle 13 is connected by shaft 15 to a ring deployment mechanism 20. Handle 13 comprises an actuating member 22 operable to communicate actuating forces to ring deployment mechanism 20. In the present example, applier 10 further includes a second actuating member 24, which is optional. Actuating members 22, 24 comprise sliders. Second slider 24 is located distal of first slider 22. Those of ordinary skill in the art will appreciate, however, that handle 13 may have any suitable number of actuating members 22, 24, that actuating members 22, 24 may take a variety of alternative forms, and that handle 13 and/or actuating members 22, 24 may be configured in a variety of alternative ways.

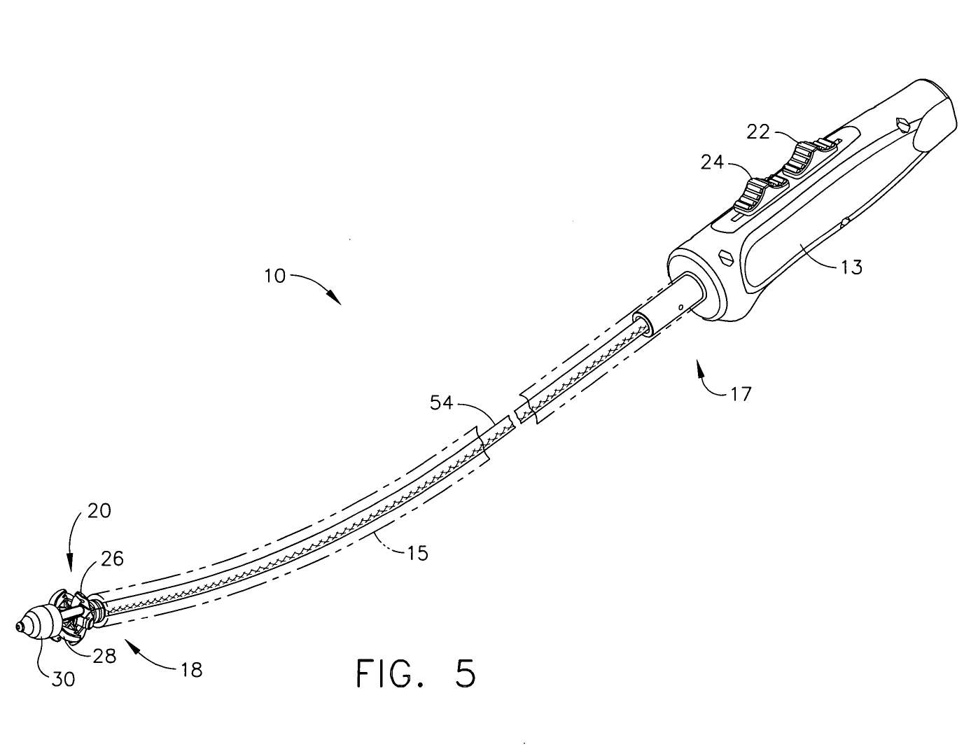

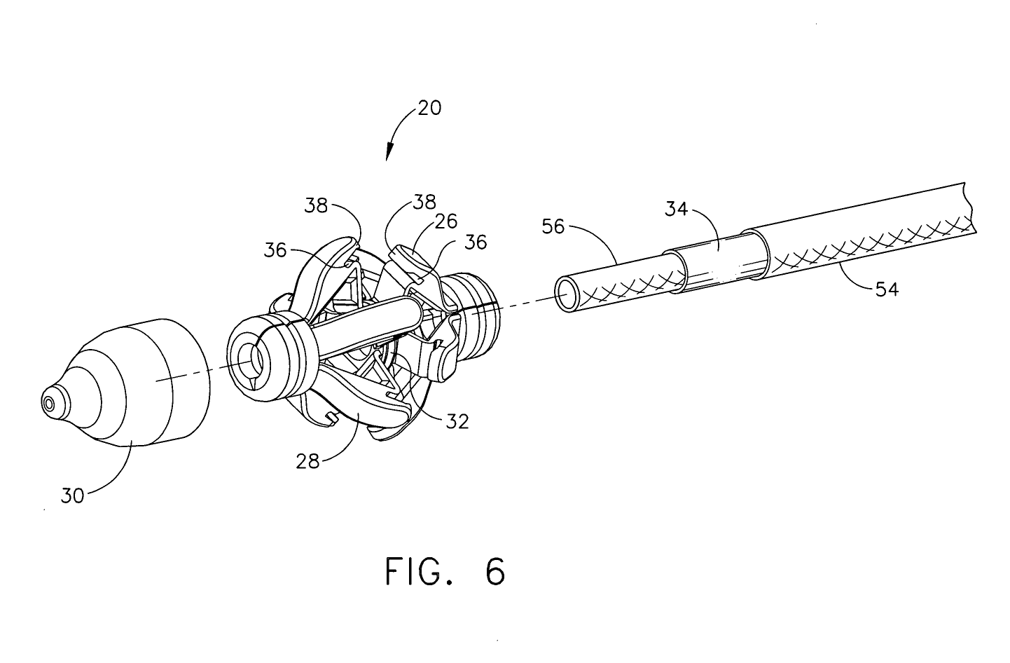

As shown in FIG. 5, sliders 22, 24 are adapted to slide from a first, unactuated position (FIG. 9) to an actuated position (FIG. 11) to actuate ring deployment mechanism 20. As best shown in FIG. 6, ring deployment mechanism 20 comprises proximal fingers 26 and distal fingers 28. Fingers 26, 28 are configured to hold an anastomotic ring by engaging petals 51 prior to and during deployment of the anastomotic ring, and release petals 51 upon deployment of the anastomotic ring. Applier 10 includes a tip 30 located distal of deployment mechanism 20. Tip 30 may attach to distal fingers 26, and/or may be connected by a rod (not pictured) to handle 13.

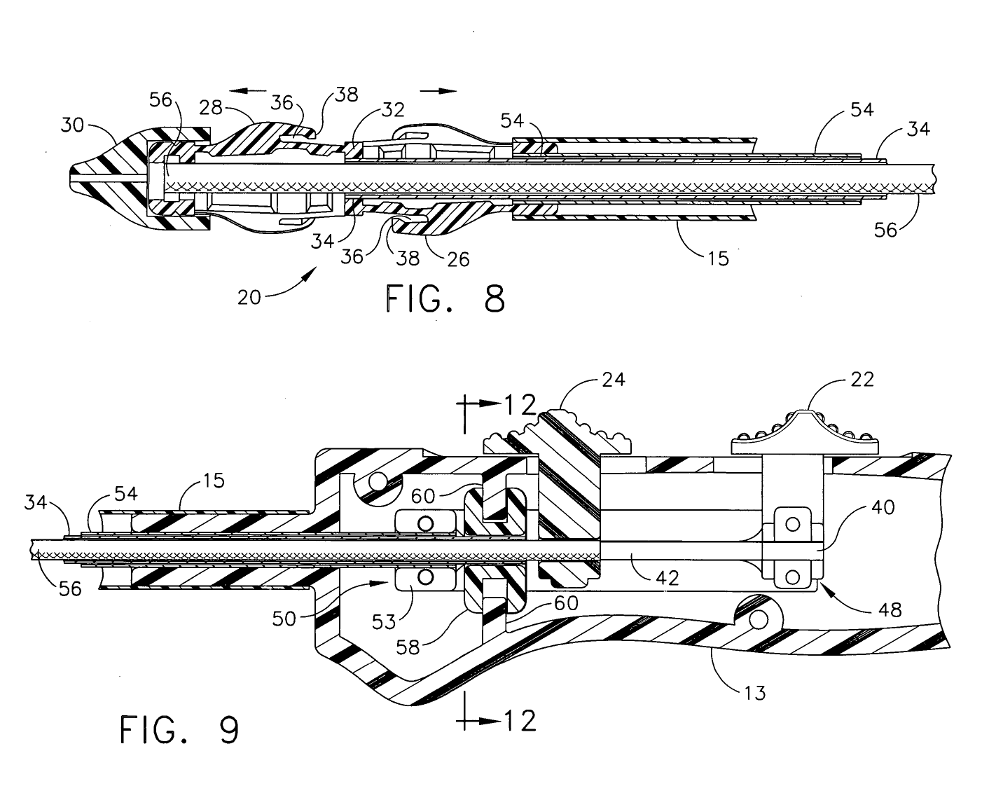

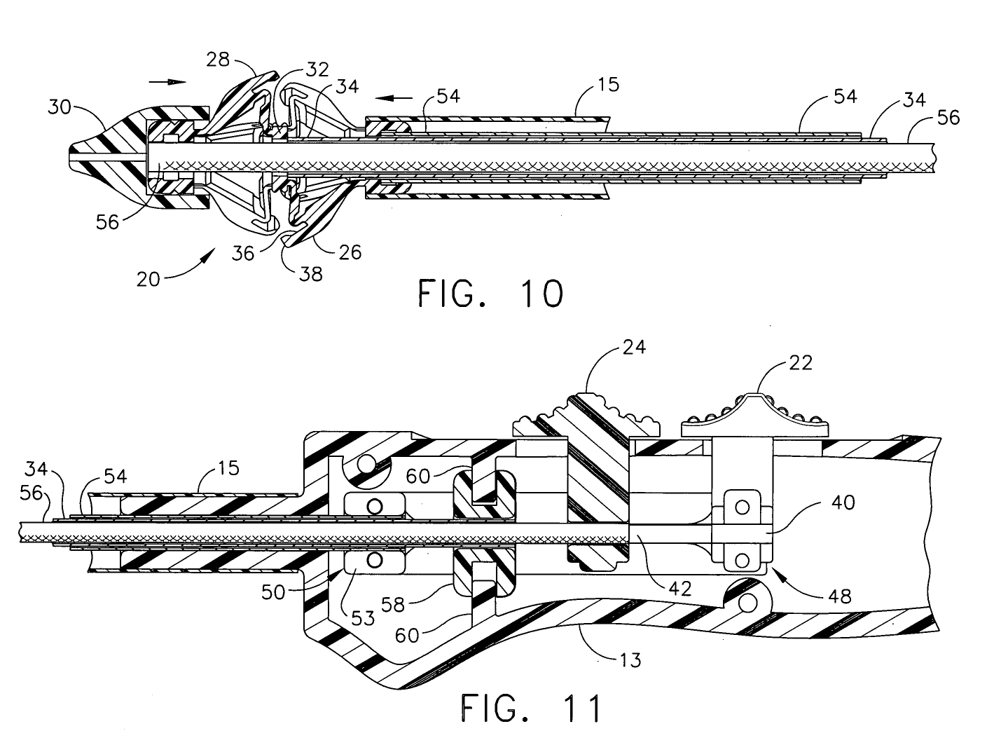

In the present example, ring deployment mechanism 20 comprises a stationary mid-ring 32 (FIG. 8). Mid-ring 32 is held stationary by a ground tube 34. Proximal and distal fingers 26, 28 are each in a double-hinged relationship with mid-ring 32. Fingers 26, 28 each comprise gripping slots 36 configured to hold the anastomotic ring prior to ring deployment, as well as during intermediate stages of ring deployment. Fingers 26, 28 further comprise inwardly-directed tips 38 configured to allow the anastomotic ring to slide out of engagement with fingers 26, 28 when the ring is fully deployed. Proximal and distal fingers 26, 28 are adapted to receive a proximal and distal portion, respectively, of an anastomotic ring. Proximal and distal fingers 26, 28 are further adapted to move from a first, unactuated position (FIG. 8) toward mid-ring 32 to a second, actuated position (FIG. 10) to deploy the anastomotic ring. As fingers 26, 28 move toward mid-ring 32, they are configured to articulate outwardly in the manner of an umbrella due to their hinged relationship with mid-ring 32, moving the anastomotic ring from the compressed position to the deployed, rivet-shaped position. Of course, ring deployment mechanism 20 may comprise a variety of alternative components and/or configurations. Such alternatives will be apparent to those of ordinary skill in the art.

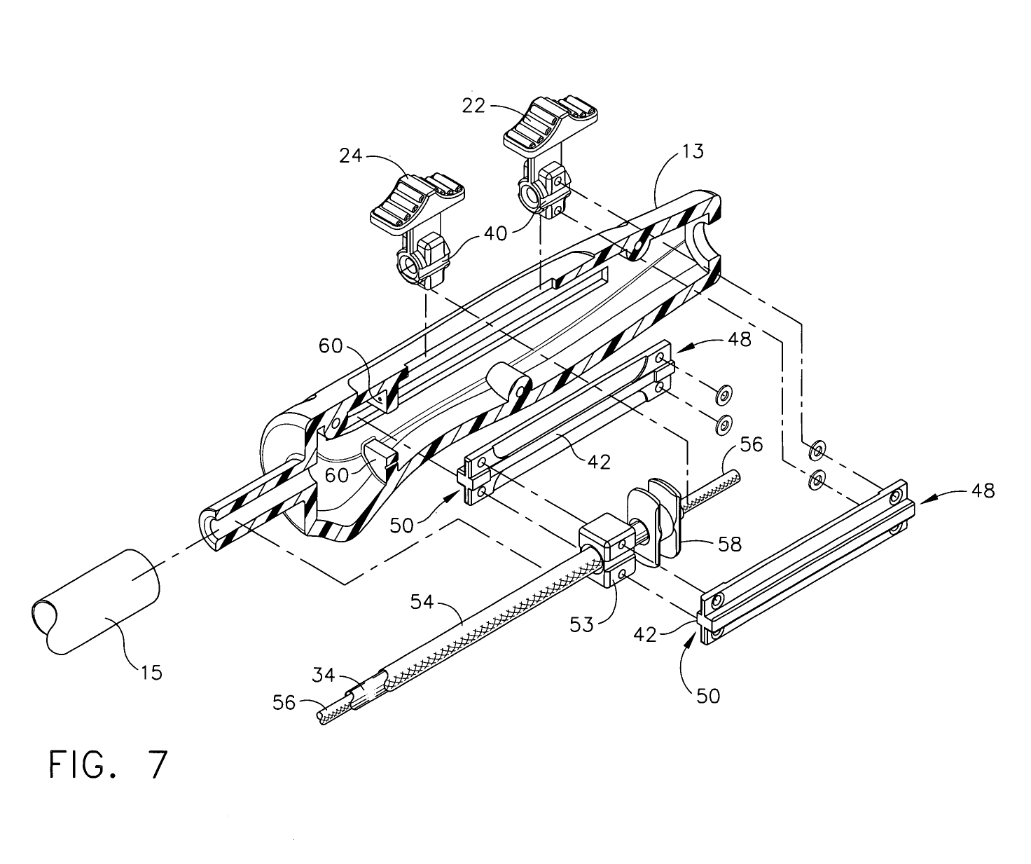

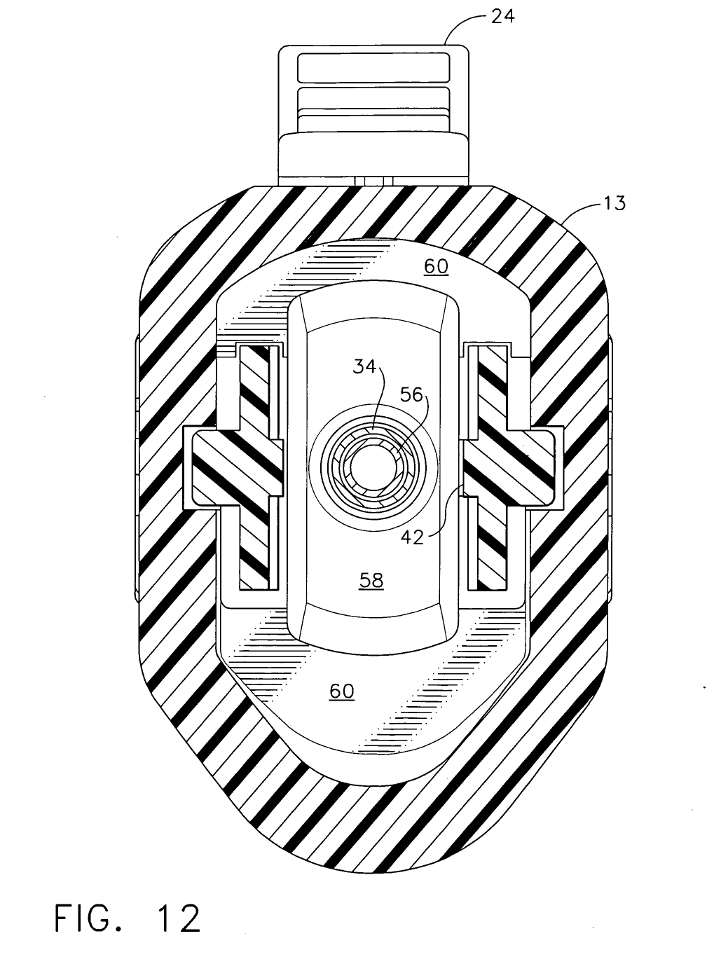

Referring now to FIGS. 7, 9, 11, and 12, a mechanism configured to transmit user input to ring deployment mechanism 20 is shown. In the present example, first deployment actuator 22 is operable to control proximal fingers 26, and second deployment actuator 24 is operable to control distal fingers 28. Alternatively, first deployment actuator 22 may control distal fingers 28 and second deployment actuator 24 may control proximal fingers 26. In the present example, first and second ring deployment actuators 22, 24 each comprise a pair of grooves 40 that are configured to slide on a track 42. Track 42 is further configured to slide within handle 13. Other suitable configurations will be apparent to those of ordinary skill in the art.

In the present example, first actuator 22 is fixedly attached to a proximal portion 48 of track 42. A distal portion 50 of track 42 is fixedly attached to a slider 53 that is slideably attached to handle 13. Slider 53 is connected to an outer tube 54. Longitudinal motion of first actuator 22 may thereby cause corresponding longitudinal motion of track 42, slider 53 and outer tube 54. Outer tube 54 is connected to proximal fingers 26. Outer tube 54 is thereby operable to communicate motion to proximal fingers 26.

Second actuator 24 is connected to an inner tube 56. Inner tube 56 extends longitudinally through ground tube 34, which extends longitudinally through outer tube 54. Inner tube 56 is connected to distal fingers 28. Inner tube 56 is thereby operable to communicate motion to distal fingers 28. In this manner, first actuator 22 is operable to control actuation of proximal fingers 26, and second actuator 24 is operable to control actuation of distal fingers 28. Ground tube 34, which is fixed to mid-ring 32 at the distal end of ground tube 34, is fixedly attached to anchor member 58 at the proximal end of ground tube 34. Anchor member 58 is configured to engage with bosses 60 in handle 13, thereby preventing relative motion between handle 13 and ground tube 34.

In the present example, it should be noted that although second actuator 24 is configured to slide on track 42, it is not statically attached to it. Therefore, longitudinal movement of track 42 due to motion of first actuator 22 will not cause longitudinal movement of second actuator 24. Those of ordinary skill in the art will appreciate, however, that a variety of alternative components and/or configurations may be used to effect actuation of distal fingers 28 and/or proximal fingers 26. By way of example only, one alternative configuration may include configuring first actuator 22 to be operable to control actuation of distal fingers 28, and configuring second actuator 24 to be operable to control actuation of proximal fingers 26. Other suitable variations will be apparent to those of ordinary skill in the art.

Ground tube 34 of the present example is rigid and comprises a curved portion. It will be appreciated that ground tube 34 is thus suitable for colorectal applications. Inner tube 56 and outer tube 54 are flexible, which allows them to follow the curve of ground tube 34 as they translate longitudinally in response to actuation of first and second actuators 22, 24. In one embodiment, inner and outer tubes 54, 56 are comprised of tightly woven wires. Other suitable features configurations of tubes 34, 54, and 56 will be apparent to those of ordinary skill in the art.

Having shown and described various embodiments and concepts of the invention, further adaptations of the methods and systems described herein can be accomplished by appropriate modifications by one of ordinary skill in the art without departing from the scope of the invention. Several of such potential alternatives, modifications, and variations have been mentioned, and others will be apparent to those skilled in the art in light of the foregoing teachings. Accordingly, the invention is intended to embrace all such alternatives, modifications and variations as may fall within the spirit and scope of the appended claims and is understood not to be limited to the details of structure and operation shown and described in the specification and drawings. Additional advantages may readily appear to those skilled in the art.

What is claimed is:

1. A surgical instrument for implanting an anastomotic ring device, comprising:
(i) a handle;
(ii) a ring deployment mechanism configured to receive an anastomotic ring, wherein the ring deployment mechanism is adapted to move between an unactuated, generally cylindrical position and an actuated, hollow rivet-forming position;
(iii) an actuation mechanism operable to communicate actuating force to the ring-deployment mechanism; and
(iv) an elongate shaft connecting the handle to the ring deployment mechanism;
wherein the elongate shaft comprises one or more rigid, curved members.

2. The surgical instrument of claim 1, further comprising an actuating member operable to actuate the ring deployment mechanism.

3. The surgical instrument of claim 2, wherein the actuating member is connected to the ring deployment mechanism by a flexible tube.

4. The surgical instrument of claim 3, wherein the flexible tube comprises woven wires.

5. The surgical instrument of claim 1, wherein the ring deployment mechanism further comprises a stationary non-actuating element.

6. The surgical instrument of claim 5, wherein the ring deployment mechanism further comprises a translating element.

7. The surgical instrument of claim 6, wherein the translating element is in a hinged relationship with the stationary non-actuating element.

8. The surgical instrument of claim 7, wherein the translating element is configured to move toward the stationary element in response to actuating force, causing the translating element to articulate outwardly from the elongate shaft to deploy a portion of the anastomotic ring.

9. A surgical instrument for implanting an anastomotic ring device, comprising:
(i) a handle;
(ii) an elongate shaft comprising a proximal portion and a distal portion, wherein the proximal portion is attached to the handle, wherein the elongate shaft comprises at least one rigid, curved member;
(iii) a ring deployment mechanism located on the distal portion of the shaft, the ring deployment mechanism comprising a first plurality of fingers configured to receive an anastomotic ring, the fingers being moveable from a first position longitudinally aligned with the shaft to a second position in which the fingers actuate outwardly from a longitudinal axis of the elongate shaft to actuate at least a portion of an anastomotic ring.

10. The surgical instrument of claim 9, wherein the first plurality of fingers are configured to receive and deploy a proximal portion of the anastomotic ring, wherein the ring deployment mechanism further comprises a second plurality of fingers configured to receive and deploy a distal portion of the anastomotic ring.

11. The surgical instrument of claim 10, further comprising a first actuating member operable to actuate the first plurality of fingers and a second actuating member operable to actuate the second plurality of fingers.

12. The surgical instrument of claim 11, wherein the first actuating member is connected to the first plurality of fingers by a first flexible tube and the second actuating member is connected to the second plurality of fingers by a second flexible tube.

13. The surgical instrument of claim 12, wherein the first and second flexible tubes are comprised of woven wires.

14. The surgical instrument of claim 13, wherein the ring deployment mechanism comprises a stationary element positioned between the first plurality of fingers and the second plurality of fingers.

15. The surgical instrument of claim 14, wherein the stationary element is held in place by the rigid, curved element.

16. A surgical instrument for implanting an anastomotic ring, comprising:
(i) a handle;
(ii) an elongate shaft comprising a proximal portion and a distal portion, wherein the proximal portion is attached to the handle, wherein the elongate shaft comprises a rigid, curved element;
(iii) a ring deployment mechanism comprising a longitudinal end and a center portion, wherein the ring deployment mechanism is positioned on the distal portion of the elongate shaft, wherein the ring deployment mechanism is configured to receive a compressed anastomotic ring; and
(iv) an actuation mechanism comprising a flexible connecting member, wherein the flexible connecting member is operable to move the longitudinal end of the ring deployment mechanism toward the center portion of the ring deployment mechanism to actuate at least a portion of an anastomotic ring.

17. The surgical instrument of claim 16, wherein the ring deployment mechanism further comprises a second longitudinal end operable to move from an initial position toward the center portion of the ring deployment mechanism to actuate a second portion of the anastomotic ring.

18. The surgical instrument of claim 17, wherein the first longitudinal end is connected to a first actuating member by the flexible connecting member.

19. The surgical instrument of claim 18, wherein the second longitudinal end is connected to a second actuating member by a second flexible connecting member.

20. The surgical instrument of claim 19, wherein the first connecting member and the second connecting member are configured to translate longitudinally within the elongate shaft.
